(19) United States
(12) Patent Application Publication
(10) Pub. No.: US 2026/0033864 A1
(43) Pub. Date: Feb. 5, 2026

(54) ANCHORING AND PUNCTURING DEVICES FOR MEDICAL PROCEDURES

(71) Applicant: EDWARDS LIFESCIENCES CORPORATION, Irvine, CA (US)

(72) Inventors: Daniel James Murray, Orange, CA (US); Michael G. Valdez, Riverside, CA (US); Jose Islas, Corona, CA (US); Arvin T. Chang, Yorba Linda, CA (US)

(21) Appl. No.: 19/356,501

(22) Filed: Oct. 13, 2025

Related U.S. Application Data
(63) Continuation of application No. PCT/US2024/024778, filed on Apr. 16, 2024.
(60) Provisional application No. 63/496,942, filed on Apr. 18, 2023.

(51) Int. Cl. A61B 17/34; A61B 17/00; A61M 25/00

(52) U.S. Cl. CPC A61B 17/3478; A61B 17/3496; A61B 2017/00292; A61B 2017/3454; A61B 2017/3488; A61M 2025/0095

(57) ABSTRACT

Puncture and anchor devices are disclosed that facilitate puncturing through a wall of a second vessel from a first vessel. The anchor can be delivered in a catheter or other delivery device and then released after puncturing through the vessel walls. The anchor can remain in the tissue, acting as a shunt. The devices and methods disclosed herein relate to a puncture device that includes a sharpened coil; a needle configured to extend from the distal end of the catheter through a central axis of the sharpened coil, wherein the sharpened coil anchors the catheter to the targeted tissue to facilitate puncturing of the targeted tissue with the needle. The needle comprises a dilation device that is configured to open the tissue and to create a conduit between both vessels. The sharpened coil is detachable from the catheter.

### CROSS-REFERENCE TO RELATED APPLICATION(S)

This application is a continuation of Int'l Pat. App. No. PCT/US2024/024778 filed Apr. 16, 2024, which claims the benefit of U.S. Prov. App. No. 63/496,942 filed Apr. 18, 2023, each of which is incorporated by reference in its entirety for all purposes.

### BACKGROUND

### Field

The present disclosure relates to the field of puncturing devices for medical procedures.

### Description of the Related Art

Certain medical procedures involve puncturing out of one vessel and into another. This can be done to accomplish a number of targeted outcomes, such as redirecting blood flow. One such procedure is a Glenn Procedure that involves connecting the superior vena cava to the right pulmonary artery. In addition, pulmonary hypertension is a rapidly deteriorating vascular disease associated with high short-term mortality rates. A primary driver of disease progression is the increase in pulmonary arterial pressure due to a reduction in vascular compliance.

### SUMMARY

Described herein are methods and/or devices that include anchoring mechanisms for needle puncture devices. The anchoring mechanisms can be used to facilitate puncturing from one vessel to another to create a fluid pathway or to provide a shunt between two vessels. The anchoring mechanisms may also be referred to as implants.

In some implementations, the anchoring mechanism can also serve as a shunt. In some implementations, the anchoring mechanism can be a sharpened coil. In some implementations, the sharpened coil facilitates puncturing of tissue and facilitates dilating the tissue. In some implementations, the sharpened coil is removed from a catheter to be implanted in tissue. In some implementations, the sharpened coil creates a conduit between vessels.

In some implementations, a puncture device includes a sharpened coil; and a needle configured to extend from the distal end of the catheter through a central axis of the sharpened coil, wherein the sharpened coil anchors the catheter to the targeted tissue to facilitate puncturing of the targeted tissue with the needle. In some implementations, the puncture device further includes a dilation device that is configured to open the tissue and to create a conduit between both vessels. In some implementations, the sharpened coil is detachable from the catheter.

In some implementations, the anchoring mechanism (or implant) can be releasably secured to a catheter. In some implementations, the implant is a stent implanted in a targeted vessel and a needle is used to puncture from a first vessel to the targeted vessel, the stent configured to induce hoop stress in the targeted vessel. In some implementations, the stent is a coil. In some implementations, the stent forms a slot feature.

In some implementations, a first flange can be implanted in a first vessel and a second flange can be implanted in a second vessel with sutures connecting the first and second flanges. The sutures can be pulled to approximate the first and second flanges to reduce the distance between the first and second vessels. A dilator can be used to dilate an opening between the first and second vessels. In some implementations, a shunt can be implanted in the dilated opening between the first and second flanges.

In some implementations, a puncture device includes a catheter configured to be torqued; a sharpened coil coupled to the distal end of the catheter; and a needle configured to extend from the distal end of the catheter approximately through a central axis of the sharpened coil. Rotating or torquing the catheter causes the sharpened coil to rotate to drive the sharpened coil into targeted tissue. The sharpened coil anchors the catheter to the targeted tissue to facilitate puncturing of the targeted tissue with the needle.

In some implementations, the puncture device further includes a dilation device that is configured to open the tissue to create a conduit between two vessels. In some implementations, the sharpened coil is detachable from the catheter. In some implementations, the puncture device further includes a release wire threaded through a proximal opening formed in the sharpened coil. In some implementations, the release wire secures the sharpened coil to the catheter by being threaded through the proximal opening. In some implementations, the sharpened coil is configured to be released from the catheter by pulling the release wire proximally to unthread the release wire from the proximal opening formed in the sharpened coil. In some implementations, the catheter includes a connection mechanism comprising a ball in contact with a spring and a pull wire coupled to the spring, the spring configured to bias a position of the ball to seat within an indentation formed in a proximal end of the sharpened coil, wherein pulling the pull wire adjusts the spring to allow the ball to move out of the indentation to release the sharpened coil.

In some implementations, the sharpened coil includes a pull wire attached to a distal end of the sharpened coil such that a proximal force on the pull wire causes the sharpened coil to compress. In some implementations, a distal end and a proximal end of the sharpened coil each have a larger radius than a middle portion of the sharpened coil between the distal end and the proximal end.

In some implementations, a puncture device includes a sharpened coil; a needle configured to extend from the distal end of the catheter through a central axis of the sharpened coil, wherein the sharpened coil anchors the catheter to the targeted tissue to facilitate puncturing of the targeted tissue with the needle; and a dilation device that is configured to open the tissue and to create a conduit between both vessels.

In some implementations, a puncture device includes a sharpened coil and a needle configured to extend from the distal end of the catheter through a central axis of the sharpened coil. The sharpened coil anchors the catheter to the targeted tissue to facilitate puncturing of the targeted tissue with the needle. The sharpened coil is detachable from the catheter.

In some implementations, a method of creating a conduit between a first vessel and a second vessel includes advancing a delivery device into the first vessel to a targeted location; deploying a stent at the targeted location in the first vessel, the stent configured to induce hoop stress in the first vessel; advancing a needle puncture device into the second vessel; and puncturing the second vessel with the needle puncture device to create a puncture from the second vessel to the first vessel, the puncturing facilitated by the hoop stress induced by the stent. The needle puncture device punctures the first vessel at the targeted location coinciding with the stent.

In some implementations, the stent forms a slot feature. In some implementations, the needle puncture device punctures through the first vessel at a location corresponding to the slot feature formed by the stent. In some implementations, the method further includes advancing a guide wire through the puncture. In some implementations, the method further includes retracting the stent such that the stent does not contact the guide wire during retraction of the stent.

In some implementations, the stent forms a coil upon being deployed. In some implementations, the stent is in a straightened configuration during advancement to the targeted location. In some implementations, the method further includes transitioning the stent to a straightened configuration after the needle puncture device punctures the first vessel. In some implementations, the method further includes retracting the stent into the delivery device in the straightened configuration.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 illustrates several access pathways for maneuvering guidewires and catheters in and around the heart to deploy the anchors, needles, and shunts disclosed herein.

FIG. 2 illustrates an example of a delivery device with a puncture needle that is puncturing through a wall of a first vessel and through a wall of a second vessel.

FIG. 3 illustrates an example anchoring and puncturing process that includes implanting a stent in a targeted vessel to induce hoop stress in the targeted vessel.

FIG. 4 illustrates an example stent that forms a slot feature.

FIG. 5 illustrates another example stent that forms a coil within a targeted vessel.

FIGS. 6A and 6B illustrate a sharpened coil driven through tissue using a rotational motion.

FIG. 7 illustrates an example of a sharpened coil implanted between a first vessel wall and a second vessel wall.

FIG. 8 illustrates an example of a sharpened coil implanted between the SVC and the RPA.

FIGS. 9A, 9B, and 9C illustrate a sharpened coil that can be used as leverage for puncturing and dilating and that can also be used as a shunt.

FIGS. 10A and 10B illustrate a coil implant that can be releasably attached to a delivery device using a release wire.

FIGS. 11A and 11B illustrate a front view and a side view, respectively, of a proximal end of a coil implant with a connection mechanism to secure the coil implant to a catheter.

FIGS. 12A, 12B, 12C, and 12D illustrate an implant connector and a locking mechanism in a locked configuration to secure an implant to a catheter body.

### DETAILED DESCRIPTION

The headings provided herein are for convenience only and do not necessarily affect the scope or meaning of the claimed subject matter.

### Overview

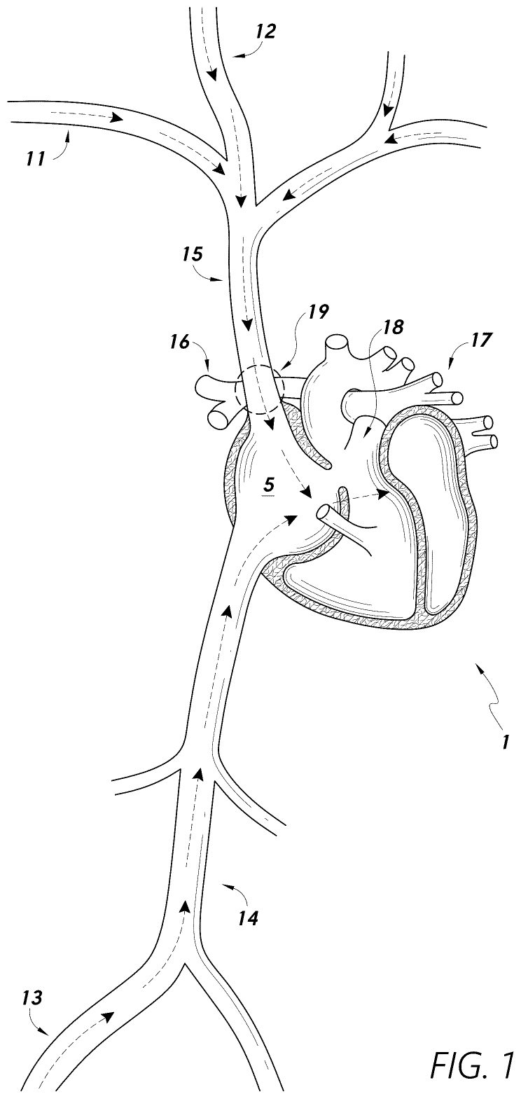

FIG. 1 illustrates several access pathways for maneuvering guidewires and catheters in and around the heart 1 to deploy the anchors, needles, and shunts disclosed herein. For instance, access may be from above via either the subclavian vein 11 or jugular vein 12 into the superior vena cava (SVC) 15, right atrium (RA) 5 and from there into the coronary sinus (CS). Alternatively, the access path may start in the femoral vein 13 and through the inferior vena cava (IVC) 14 into the heart 1. Other access routes may also be used, such as through the pulmonary artery 18, with each access route typically utilizing a percutaneous incision through which the guidewire and catheter are inserted into the vasculature, normally through a sealed introducer. From there, the physician controls the distal ends of the devices from outside the body.

The pulmonary artery 18 branches into a right pulmonary artery (RPA) 16 and a left pulmonary artery (LPA) 17. Some examples of the present disclosure may involve delivering one or more implants (e.g., shunts) to an intersection 19 and/or crossing point between the RPA 16 and the SVC 15. For example, the RPA 16 may extend generally perpendicularly to the SVC 15 and/or can cross behind/in front of the SVC 15. In some cases, the RPA 16 may contact the SVC 15 while in other cases there may be a separation between the RPA 16 and the SVC 15.

Some examples described herein involve delivering a shunt system percutaneously to connect the RPA 16 with the SVC 15. Given that the RPA 16 and SVC 15 are adjacent anatomically, the intersection 19 area of the two provides a desirable location to establish a shunt. Further, because the RPA 16 has higher pressures than the SVC 15, particularly under pulmonary hypertensive conditions, unidirectional movement of blood flow is consistently diverted out of the RPA 16 and into the SVC 15. The net result of this shunting is to decompress and lower the pressure in the main pulmonary artery 18, including mean and peak systolic pressure. This is turn reduces the afterload on the right ventricle and reduces the amount of work required to eject blood, thereby decreasing right ventricle compensatory responses to pulmonary hypertension and preserving ventricular-vascular coupling. Shunting in some examples herein may occur at all pressures, or a shunt system may be pre-loaded to dynamically shunt at an offset pressure.

In cases when an interventional medical procedure dictates a puncture out of one vessel into another vessel, for example, a procedure to place a shunt between the Right Pulmonary Artery (RPA) and Superior Vena Cava (SVC) in the heart, puncturing into the second vessel can be difficult. While the disclosure herein focuses on connecting and/or shunting between the RPA 13 and the SVC 15, this is for illustrative purposes and the examples described herein can be applied to other areas of anatomy. Puncturing out of the first vessel is easier in comparison, because devices inside the first vessel can be deflected and anchored in the first vessel and used as leverage to puncture out. Additionally, the deflection of the device in the first vessel can induce hoop stress in the first vessel, causing it to be more susceptible to puncturing.

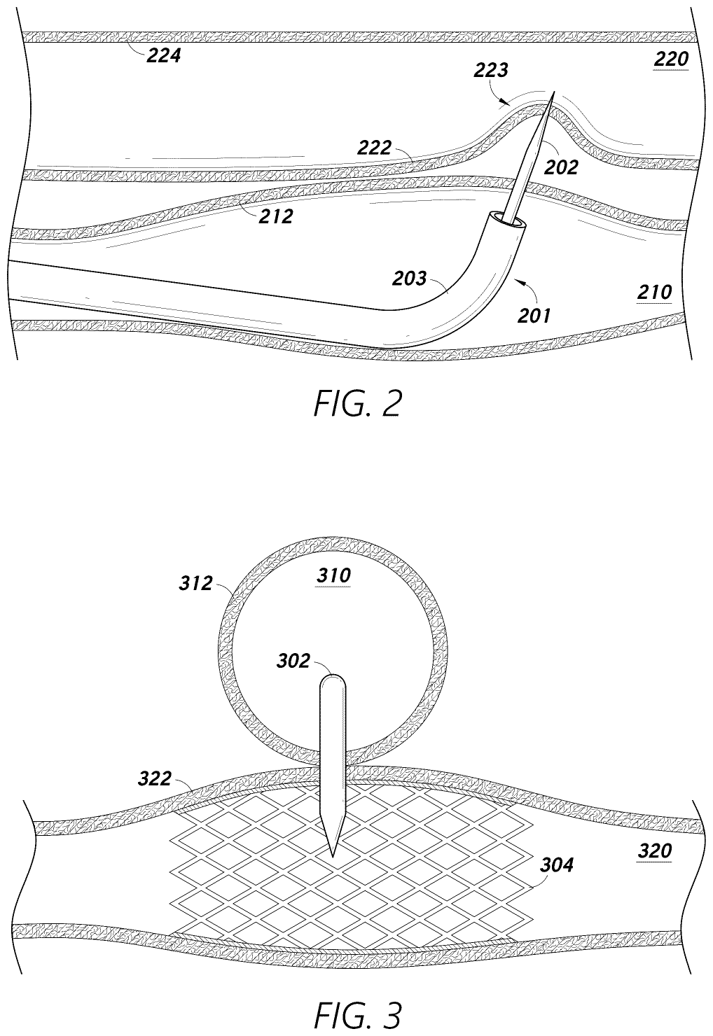

FIG. 2 illustrates an example of a delivery device 201 with a puncture needle 202 that is puncturing through a wall 212 of a first vessel 210 and through a tissue wall 222 of a second vessel 220. Puncturing through the first vessel wall 212 is facilitated by the body 203 of the delivery device 201 providing apposition to the puncture needle 202. The second vessel 220 does not have any leverage point or induced stress, so the puncture needle 202 meets the tissue and “tents” the tissue (the tented tissue 223) without puncturing across the tissue wall 222. If excessive force is exerted, puncture of the second vessel 220 can occur, but at a risk of the puncture needle 202 jumping abruptly to the opposite side wall 224 with a risk of puncturing through the entire second vessel 220.

To address these and other issues, disclosed herein are puncture and anchor devices that facilitate puncturing through a wall of a second vessel from a first vessel. In some implementations, the anchor can be delivered in a catheter or other delivery device and then released after puncturing through the vessel walls. The anchor can remain in the tissue, acting as a shunt for example. This is different from other puncture devices that do not have an additional anchoring mechanism to allow for puncturing out of one vessel into another.

Devices disclosed herein relate to puncturing devices that are configured to puncture from one vessel into another vessel. Certain implementations of the disclosed puncturing devices include a sharpened coil attached to the distal end of a catheter. The sharpened coil is configured to be driven through both vessels. The sharpened coil is also configured to provide an anchor or leverage point for a needle to be driven through both vessels (e.g., to puncture out of one vessel and into another vessel). This can be done to place a shunt between the vessels, for example. Certain implementations of the disclosed puncturing devices include implanting devices in a targeted vessel that are configured to induce hoop stress in the targeted vessel to facilitate puncturing from a first vessel to the targeted vessel.

Some transcatheter processes described herein can utilize a single catheter or multiple catheters. The one or more catheters can be delivered via a transjugular, brachial, subclavian, and/or transfemoral approach. Within each access point, either one or two catheters may be used for the delivery. Where two catheters are used, a first catheter may comprise a stent snare catheter and/or a second catheter can comprise a puncture delivery catheter, or vice versa. In the case of a single catheter system, a puncture may be made and/or one or more implants delivered in one direction using the same catheter, in either SVC 15 to RPA 16 or vice versa directions. In some cases, it can be easier to deliver a catheter through the RPA 16 than the SVC 15 and/or IVC 14.

While certain example delivery methods are described, other delivery methods may be used for the example devices described herein. For example, a single catheter may be delivered via the SVC 15 and/or the IVC 14. In another example, a first catheter and/or a second catheter may be delivered via the RPA 16 to the SVC 15 to allow one or more shunt devices and/or puncture devices to be delivered from the SVC 15 to the RPA 16. In some implementations, a first and/or second catheter may be delivered via the SVC 15 to the RPA 16 to allow one or more shunt devices to be delivered from the RPA 16 to the SVC 15.

### Inducing Hoop Stress in a Targeted Vessel

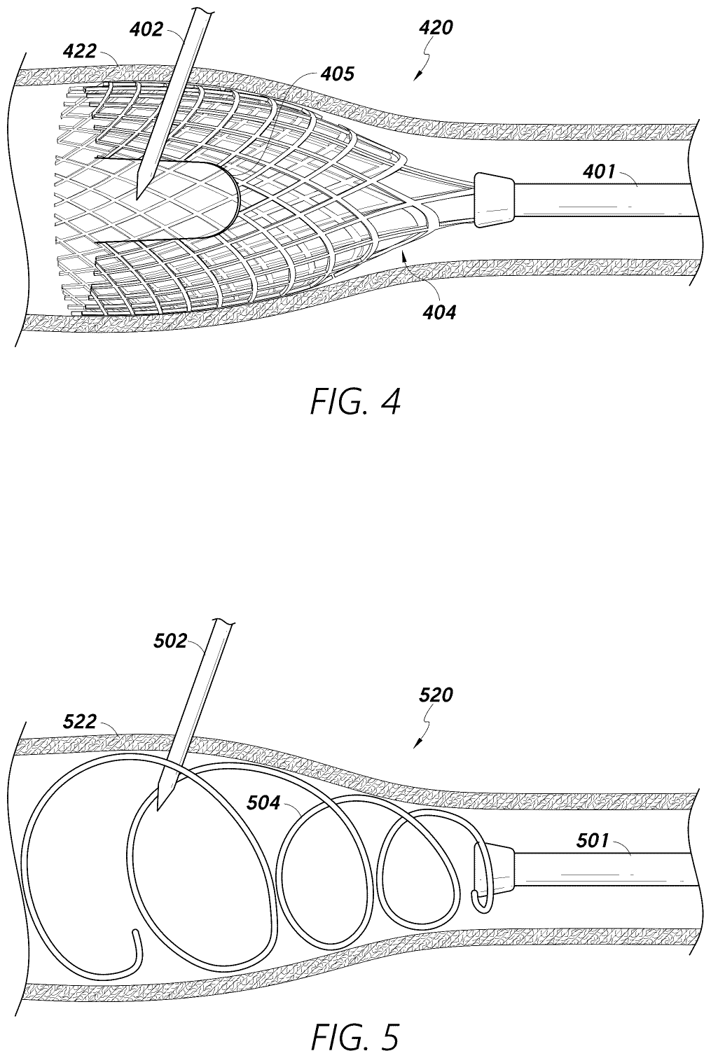

FIG. 3 illustrates an example anchoring and puncturing process that includes implanting a stent 304 in a targeted vessel 320 to induce hoop stress in the targeted vessel 320. The stent 304 can be placed in the second or targeted vessel 320, such as the right pulmonary artery or RPA. The stent 304 can be expanded to induce hoop stress in the targeted vessel 320. The hoop stress facilitates receiving a puncture from a puncturing needle 302 passing through a wall 312 of a first vessel 310, such as the superior vena cava or SVC, and through a wall 322 of the second vessel 320.

To implant the stent 304, the stent 304 can be delivered to the targeted vessel 320 using a transcatheter approach. Once in a desired location in the targeted vessel 320, the stent 304 can be deployed and expanded. The desired location can be determined using suitable imaging devices and technology, including fluoroscopy. The radius of the stent 304 can be larger than the inner diameter of the targeted vessel 320 at the desired location. Thus, when expanded, the stent 304 pushes on the walls of the targeted vessel 320 thereby inducing hoop stress. As a result, deploying the stent 304 in the targeted vessel 320 allows the targeted vessel 320 to be punctured more easily due to the induced hoop stress from the outward radial force of the stent 304.

Puncturing the targeted vessel 320 can be accomplished by directing a puncturing device with the puncturing needle 302 to a desired location in the first vessel 310. Once in the desired location, the puncturing needle 302 can be deployed to puncture through the wall 312 of the first vessel and through the wall 322 of the targeted vessel 320. In some implementations, the puncturing needle 302 can be directed to puncture through the wall 322 of the targeted vessel 320 at an open space of a cell of the stent 304. For example, the puncturing needle 302 punctures the wall 312 at a location that coincides with the stent 304. The desired location in the first vessel 310 can be determined using suitable imaging devices and technology, including fluoroscopy.

The stent 304 can be self-expanding, e.g., using a shape set memory alloy such as Nitinol. The stent 304 can be a balloon-expandable stent that can be made from, for example, a cobalt chromium frame or similar material. In some implementations, the stent 304 can be permanently deployed. In some implementations, the stent 304 can be removed as part of the medical procedure, after the targeted vessel 320 has been punctured. For example, the stent 304 can be partially deployed to facilitate the puncture of the targeted vessel 320, and then the stent 304 can be recaptured and removed.

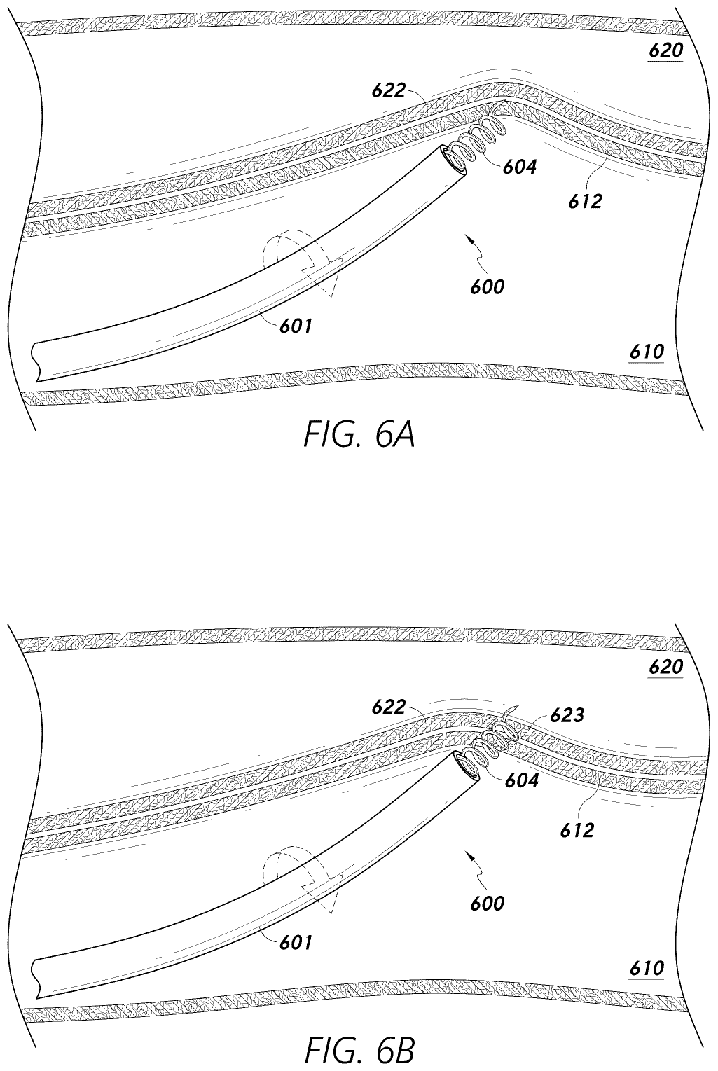

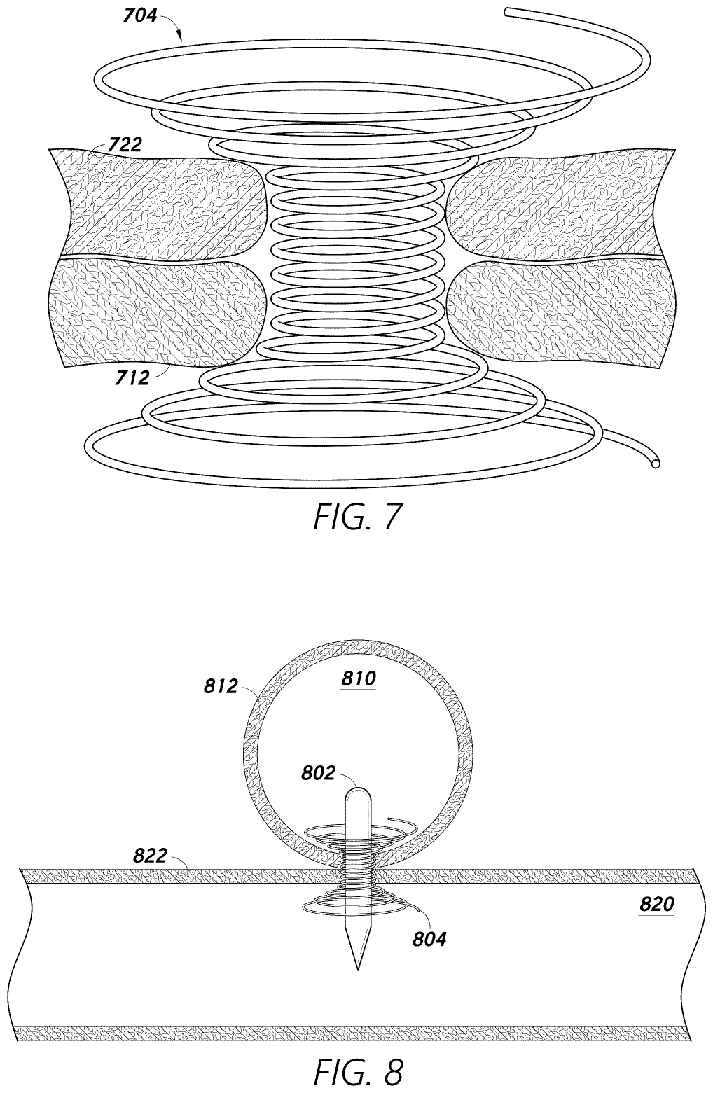

For implementations in which the stent is to be recaptured and removed, a problem may exist in which the cells of the stent interfere with a guidewire or other component deployed after puncturing the targeted vessel. Accordingly, FIGS. 4 and 5 illustrate implantable devices configured to induce hoop stress in a targeted vessel and that are also configured to avoid interfering with a guidewire or other such component when being recaptured and removed. Thus, the implantable devices of FIGS. 4 and 5 provide a feasible and practicable method for retrieving the implantable devices after deployment of a guidewire or other similar component.

FIG. 4 illustrates an example stent 404 that forms a slot feature 405. The slot feature 405 provides a landing zone space in imagery, such as fluoroscopic imagery, for a puncturing needle 402 to puncture through the wall 422 of the targeted vessel 420. Because it is a slot, once the puncture has been performed and the guidewire is inserted into the targeted vessel 420, the stent 404 can then be removed and recaptured.

For example, a delivery device 401 can deploy the stent 404 with the slot feature 405 in the targeted vessel 420. With the stent 404 deployed and inducing hoop stress in the targeted vessel 420, the puncturing needle 402 can be deployed from a different vessel to puncture through the wall 422 of the targeted vessel 420. The puncturing needle 402 can be directed to puncture through the wall 422 of the targeted vessel 420 within the slot feature 405 of the stent 404. After puncturing, a guidewire or other component can be deployed through the puncture location in the targeted vessel 420. With the guidewire deployed, the stent 404 can be retracted into the delivery device 401. This is configured to avoid interfering with the guidewire or other such component due at least in part to the guidewire being deployed through the slot feature 405. Accordingly, upon retrieval into the delivery device 401, the stent 404 is pulled back into the delivery device 401 and the guidewire is not pulled back with the stent 404 due at least in part to the slot feature 405.

FIG. 5 illustrates another example stent 504 that forms a coil within a targeted vessel 520. The stent 504 can be implanted using a delivery device 501. The coiled stent 504 can be configured to coil to expand its radius upon deployment to induce hoop stress on the targeted vessel 520. The coiled stent 504 can be created with a shape set memory alloy, such as Nitinol, and can be created so that the coiled stent 504 straightens to enable retracting the stent 504 back into the delivery device for recapture and removal.

For example, the delivery device 501 can deliver the stent 504 in a straightened configuration to the targeted vessel 520. The delivery device 501 can deploy the stent 504 whereupon the stent 504 coils in such a way as to induce hoop stress in the targeted vessel 520. Once the stent 504 has been deployed, a puncturing needle 502 can be deployed from a different vessel to puncture through the wall 522 of the targeted vessel 520. The puncturing needle 502 can be directed to puncture through the wall 522 of the targeted vessel 520 so as to not contact a portion of the coiled stent 504. After puncturing, a guidewire or other component can be deployed through the puncture location in the targeted vessel 520. With the guidewire deployed, the stent 504 can be straightened in preparation to be retracted into the delivery device 501. This is configured to avoid interfering with the guidewire or other such component due at least in part to the straightening of the coils so that no portion of the stent 504 entraps or otherwise contacts the guidewire. Accordingly, upon retrieval into the delivery device 501, the stent 504 is straightened and pulled back into the delivery device 501 and the guidewire is not pulled back with the stent 504 due at least in part to the stent 504 being in a straightened configuration rather than a coiled configuration.

Advantageously, the devices of FIGS. 3-5 provide a supporting implantable device in a targeted vessel to facilitate a puncture into the targeted vessel. This may be different from other puncturing processes or devices that do not permanently or partially deploy an implantable device in the targeted vessel to induce hoop stress in the targeted vessel.

### Coils to Provide an Anchor or Leverage Point

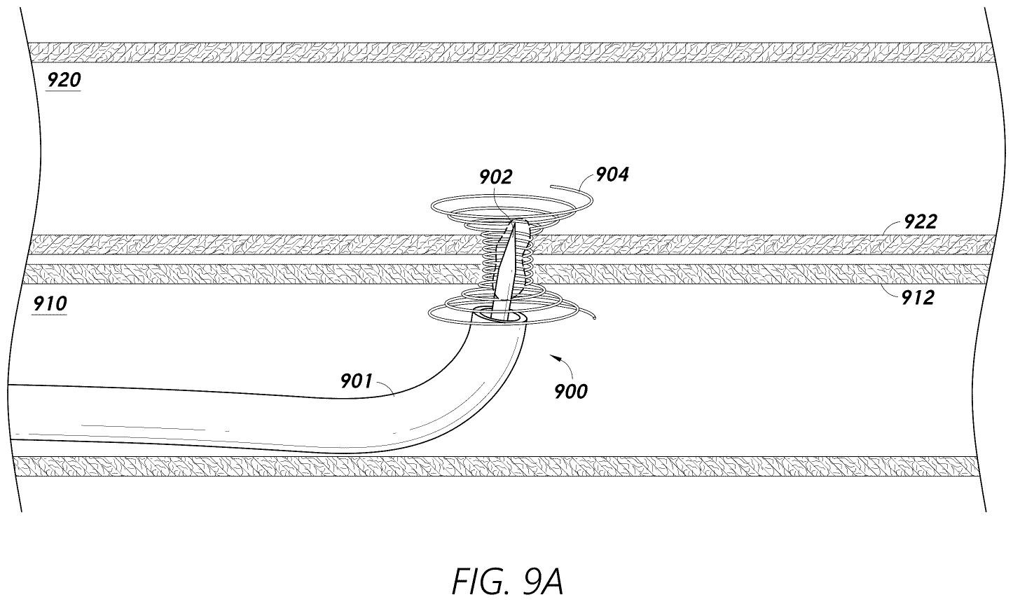

FIGS. 6A and 6B illustrate an example of a disclosed puncturing device 600. The puncturing device 600 includes a sharpened coil 604 attached to a distal end of a torqueable catheter 601. FIG. 6A illustrates that the sharpened coil 604 is driven through a rotational motion, similar to a drill, as illustrated by the arrow. The sharpened coil 604 penetrates a wall 612 of a first vessel 610 and into a wall 622 of a second vessel 620 via a rotational motion while an operator simultaneously provides axial translation. FIG. 6B illustrates the puncturing device 600 penetrating out of the first vessel 610 and into the second vessel 620 at a location 623.

Once the sharpened coil 604 has penetrated into the second vessel 620, a needle can be passed through the center or internal diameter of the coil. The needle can be configured to extend from the distal end of the catheter 601 approximately through a central axis of the sharpened coil 604. The needle can be used to puncture to create a pathway from the first vessel 610 to the second vessel 620. The sharpened coil 604 can be used as a leverage point to puncture through both vessels 610, 620. Thus, the puncturing device 600 can use the sharpened coil 604 to avoid tenting the tissue when puncturing between vessels by using the sharpened coil 604 to hold the tissue or vessels together while the needle or other puncturing element punctures through the tissue.

The puncturing device 600 can be configured to secure the sharpened coil 604 while the puncturing device 600 rotates to enable the sharpened coil 604 to penetrate the walls 612, 622 (similar to a screw). In some implementations, the catheter 601 includes a sheath that covers the sharpened coil 604 during delivery. Once at the desired puncture site, the sheath can partially reveal the sharpened coil 604 such that a portion of the sheath contacts a portion of the sharpened coil 604 to secure the sharpened coil 604 to the catheter 601. In this configuration, the proximal end of the sharpened coil 604 is cinched to the catheter to provide the ability to apply torque to the sharpened coil 604 by rotating the catheter 601. Once the sharpened coil 604 is in place (penetrating the tissue comprising the first vessel wall 612 and the second vessel wall 622), the sheath can be retracted further to disengage the sharpened coil 604 from the catheter 601. In this way, the sharpened coil 604 can be removed from the catheter 601 and left in place in the vessel walls 612, 622. In some implementations, the sharpened coil 604 can act as a shunt, examples of which are described herein. In some implementations, the catheter 601 that drives the sharpened coil 604 can be used to puncture through one or through many layers of tissues and vessels to provide an anchor or leverage point for needle puncture.

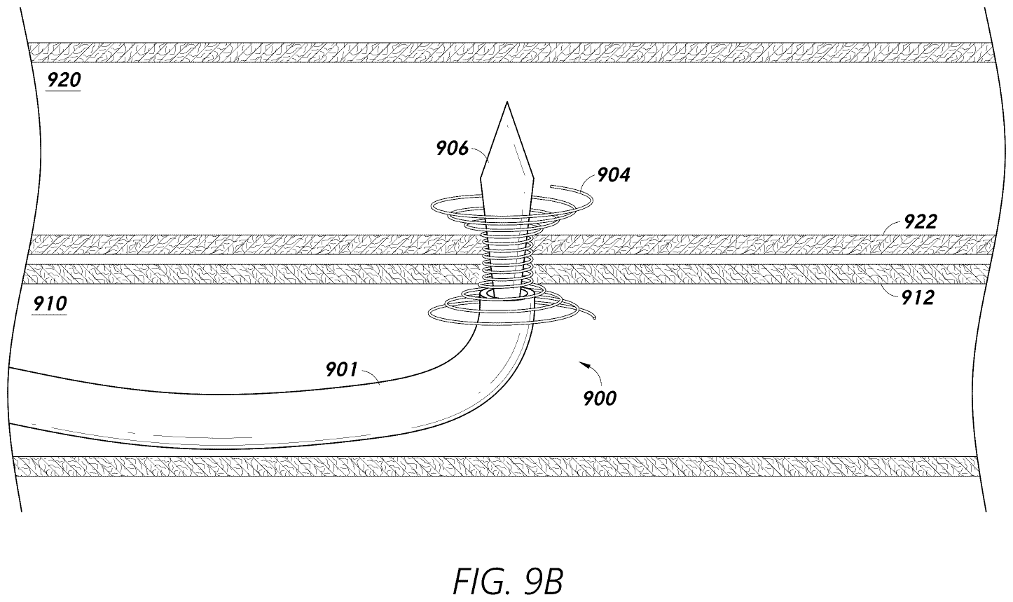

FIG. 7 illustrates an example of a sharpened coil 704 (similar to the sharpened coil 604 of FIGS. 6A and 6B) implanted between a first vessel wall 712 and a second vessel wall 722. The sharpened coil 704 can be configured to provide an anchor point for a puncturing device to facilitate puncturing through the first vessel wall 712 and the second vessel wall 722. In some implementations, the sharpened coil 704 can act as a shunt to provide a fluid pathway from a first vessel to a second vessel. In some implementations, the sharpened coil 704 can provide an anchor for a puncturing and/or dilating mechanism to create a fluid pathway from a first vessel to a second vessel. In such implementations, a shunt can be implanted in the fluid pathway created using the sharpened coil 704. In certain implementations, the sharpened coil 704 can remain implanted with any shunt or other implant. In various implementations, the sharpened coil 704 can be removed after implantation of a shunt or other such implant. Thus, the sharpened coil 704 can be configured to facilitate puncturing through tissue, dilating punctured tissue, and/or creating fluid pathways.

The sharpened coil 704 can be at least partially composed of any suitable material(s), which can include expandable stainless steel, cobalt chromium, textiles, Nitinol, braided materials (which may include stainless steel, Nitinol, and/or other metals), polymers, and/or textile materials, including flexible and/or braided textiles. Textile materials can include memory-formed textiles. The sharpened coil 704 can include fabric or textile materials at least on an inner portion or central portion of the sharpened coil 704 to promote tissue growth. This may be particularly advantageous where the sharpened coil 704 is implanted and acts as a shunt or an anchoring mechanism for a shunt.

In some implementations, the sharpened coil 704 includes coils with larger radii near end points of the sharpened coil 704 with coils having smaller radii near a middle or central portion of the sharpened coil 704. In such implementations, the coils with the larger radii can act to anchor the sharpened coil 704 in the tissue. Similarly, in such implementations, the coils with the smaller radii can act as a shunt by providing a fluid pathway through the first vessel wall 712 and the second vessel wall 722.

The sharpened coil 704 can be implanted similar to the sharpened coil 604 described herein with reference to FIGS. 6A and 6B. In some implementations, a delivery device includes a suture or other such pull wire coupled to a distal end of the sharpened coil 704. After delivery and/or implantation of the sharpened coil 704, the suture can be pulled proximally to compress the sharpened coil 704 or to approximate the coils of the sharpened coil 704 to reduce the pitch of the sharpened coil 704. This can be done to improve the fluid pathway that is provided by the sharpened coil 704, for example. This can be done once dilation has been completed (e.g., using a dilator or balloon as described herein). Thus, while the conduit created by the needle 902 is dilated, the sharpened coil 704 can be compressed to improve the fluid pathway.

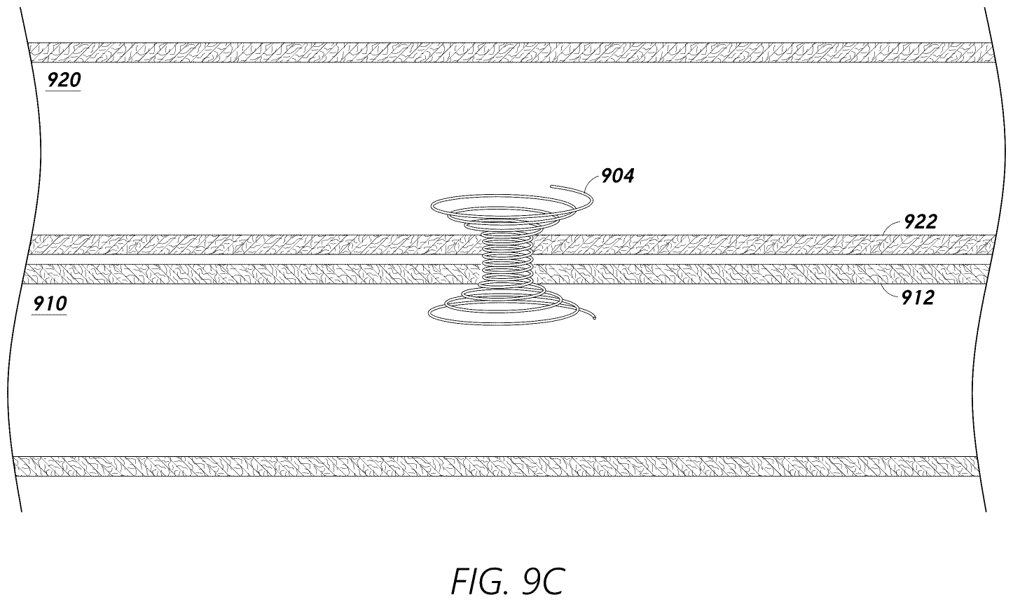

FIG. 8 illustrates an example of a sharpened coil 804 implanted between the SVC 810 and the RPA 820. The sharpened coil 804 can be implanted similar to the sharpened coil 604 described herein with reference to FIGS. 6A and 6B. The sharpened coil 804 provides an anchor point for a needle 802 configured to puncture between the SVC 810 and the RPA 820. The sharpened coil 804 is rotated and driven into the wall 812 of the SVC 810 so that it penetrates through the wall 822 of the RPA 820. The needle 802 is driven through the middle of the sharpened coil 804 to create a puncture between the SVC 810 and the RPA 820. Once the puncture is created, a shunt can be implanted in the puncture through the middle of the sharpened coil 804 or the sharpened coil 804 can act as a shunt.

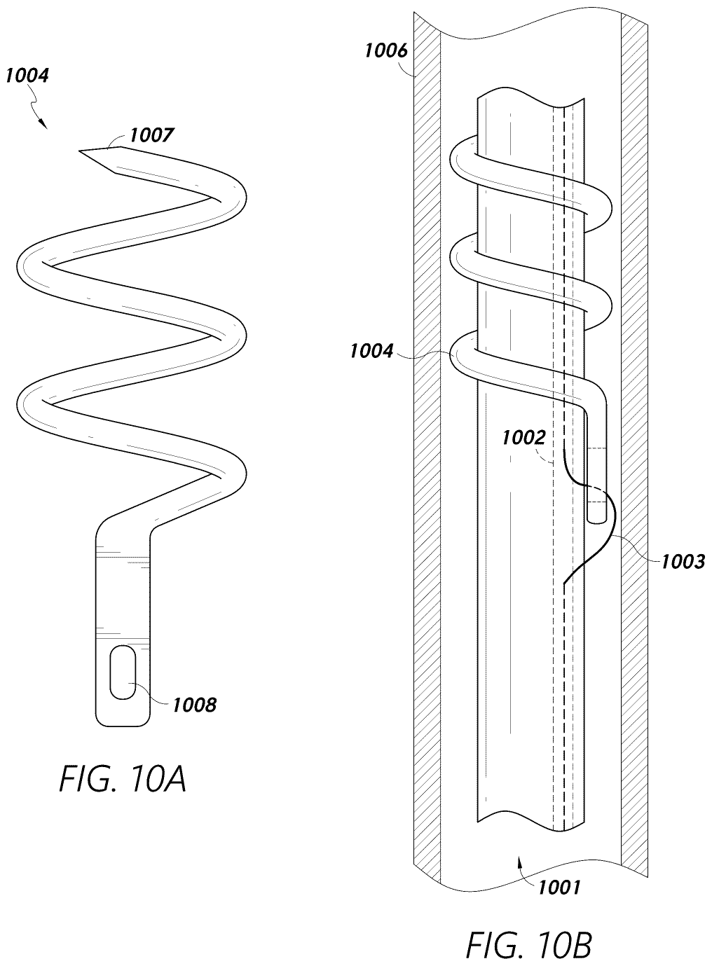

FIGS. 9A, 9B, and 9C illustrate a process for implanting a sharpened coil 904 to facilitate puncturing between vessels and dilating the puncture, the sharpened coil 904 configured to be implanted to act as a shunt between the vessels. FIG. 9A illustrates that the sharpened coil 904 can be used as leverage to pull against the tissue walls 912, 922 while simultaneously pushing a needle 902 through the center to puncture the tissue walls 912, 922. The sharpened coil 904 is similar to the sharpened coil 604 and the sharpened coil 704 described herein with reference to FIGS. 6A-8. The sharpened coil 904 can be implanted using a catheter 901, similar to the sharpened coil 604 described herein with reference to FIGS. 6A and 6B. The sharpened coil 904 can be part of a puncture device 900 that also includes the needle 902 and the catheter 901.

FIG. 9B illustrates that a dilation device 906 can be driven through the center of the sharpened coil 904 to open the tissue and to create a conduit between both vessels 910, 920. Similar to the functionality provided for the needle 902, the sharpened coil 904 can act as a leverage point for dilating the punctured tissue walls 912, 922 with the dilation device 906. In some implementations, the dilation device 906 is delivered with the same catheter 901 as the needle 902.

FIG. 9C illustrates that, after puncturing and dilating the tissue walls 912, 922, the sharpened coil 904 can be detached and left in the tissue walls 912, 922. In such implementations, the sharpened coil 904 can act as a shunt implant between both vessels 910, 920. In certain implementations, the sharpened coil 904 can include a cloth fabric sewn to the center inner diameter to encourage additional tissue ingrowth and to provide a better seal. Accordingly, an operator can deliver the sharpened coil 904 to a targeted site to create a shunt between the two vessels 910, 920, screw the sharpened coil 904 into the tissue walls 912, 922 so that the sharpened coil 904 punctures the tissue walls 912, 922, use the dilation device 906 to expand the opening created by the needle 902, and release the sharpened coil 904 to make the sharpened coil 904 serve as a shunt implant.

In some implementations, the dilation method can be replaced with a balloon to expand the orifice created by the needle 902 rather than using the dilation device 906. Using a balloon may be advantageous due at least in part to the dilation device 906 having a fixed size (e.g., the size of the dilater) whereas a balloon can over expand. Thus, the sharpened coil 904 can act as an anchor for the dilation procedure either with a dilater or a balloon.

Example Implants with Release Mechanisms

As described herein, coil implants or shunts can be anchored or driven into tissue to facilitate puncturing between vessels and/or dilating a fluid pathway between vessels. After facilitating puncturing and/or dilating, the coil implant can be released from a delivery device, such as a catheter. Any of a number of suitable mechanisms can be used to releasably attach the coil implant to the delivery device, examples of which are described herein. It should be noted that any of the releasable connection mechanisms described herein can be used with any of the coil implants described herein.

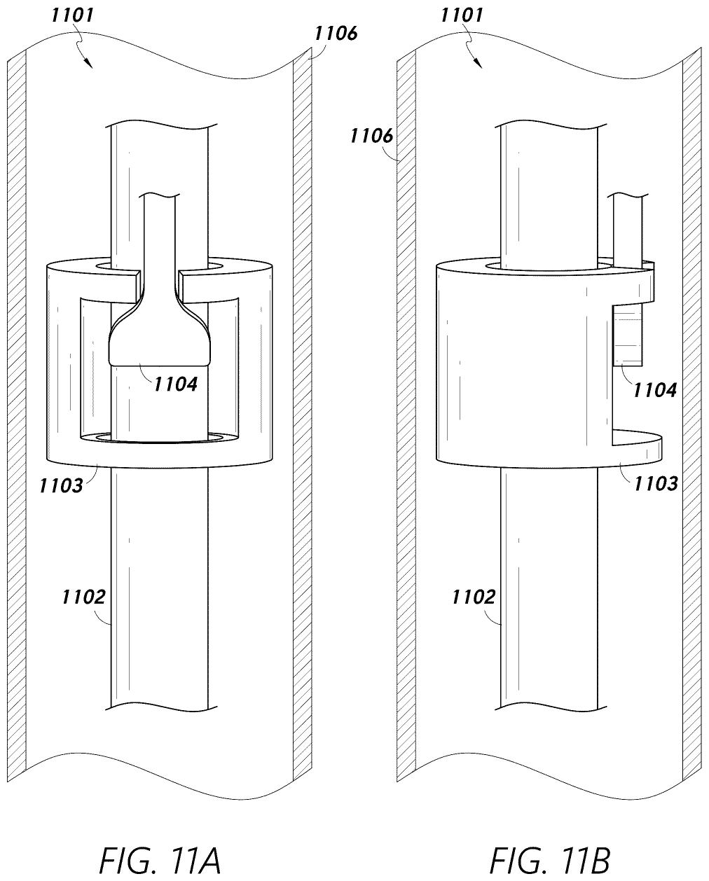

FIGS. 10A and 10B illustrate an example coil implant 1004 that can be used to anchor puncturing and/or dilating mechanisms (such as needles, dilators, balloons, etc.). FIG. 10A illustrates a front view of the coil implant 1004 when the coil implant 1004 is not attached or coupled to a delivery device. The coil implant 1004 includes a sharpened tip 1007 to facilitate puncturing tissue, as described herein. FIG. 10B illustrates the coil implant 1004 in a side view attached to a catheter 1001. The coil implant 1004 can be crimped onto the catheter 1001 for delivery. The catheter 1001 can be a multi-lumen catheter with an outer sheath 1006 and an inner lumen 1002 through which a release wire 1003 runs.

The release wire 1003 is threaded through an opening 1008 formed near a proximal end of the coil implant 1004. Thus, the release wire 1003 runs from a proximal end of the catheter 1001 through the inner lumen 1002 near an attachment point for the coil implant 1004 where it exits the inner lumen 1002 to be threaded through the opening 1008 after which it returns to the inner lumen. Threading the release wire 1003 through the opening helps to secure the coil implant 1004 to the catheter 1001 and resists relative rotational movement between the catheter 1001 and the coil implant 1004. The release wire 1003 can be tightened to lock the coil implant 1004 to the catheter 1001. The release wire 1003 is configured to be pulled to release the coil implant 1004 from the catheter 1001. In other words, the release wire 1003 can be pulled proximally to unthread the release wire 1003 from the proximal opening formed by the coil implant 1004. Thus, the release wire 1003 can be configured to secure the coil implant 1004 to the catheter 1001 during implantation. Once implanted, the release wire 1003 can be retracted to detach the coil implant 1004 from the catheter 1001.

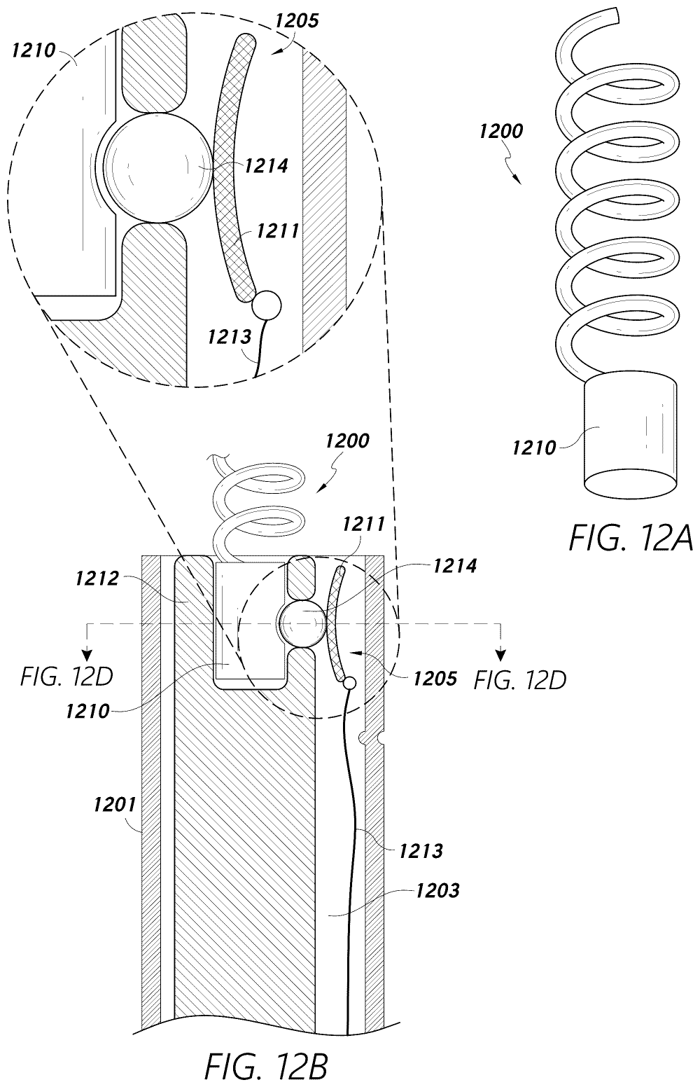

FIGS. 11A and 11B illustrate a front view and a side view, respectively, of a proximal end of a coil implant 1104 with a connection mechanism 1103 to secure the coil implant 1104 to a catheter body 1102 of a catheter 1101. The coil implant 1104 and the catheter are illustrated within a vessel 1106. The proximal end of the coil implant 1104 can be connected to the catheter body 1102 of the catheter 1101 with the connection mechanism 1103 that is cut to receive a spatula-shaped proximal leg of the coil implant 1104. To release the coil implant 1104, the proximal leg can be rotated to release from the connection mechanism 1103 and/or the connection mechanism can open to release the proximal leg.

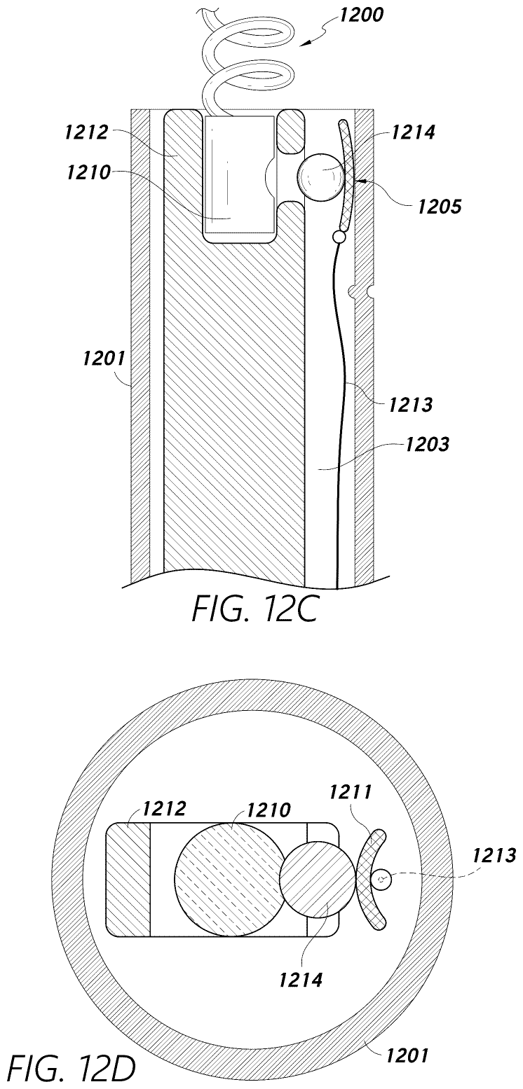

FIGS. 12A, 12B, 12C, and 12D illustrate an example implant connector 1210 for an implant 1200, such as the coil implants described herein. FIG. 12A illustrates an example of the implant and implant connector 1210. FIG. 12B illustrates a side view of the implant connector 1210 and a locking mechanism 1205 in a locked configuration to secure the implant 1200 to a catheter 1201. The implant connector 1210 mates with a complementary connector receptacle 1212 that is part of the catheter 1201. FIG. 12C illustrates a cross-sectional side view of the implant connector 1210 and the locking mechanism 1205 in an unlocked configuration to release the implant 1200 from the catheter 1201. FIG. 12D illustrates a top cross-section view of the catheter 1201 and the implant connector 1210.

A proximal end of the implant 1200 forms the implant connector 1210, the implant connector 1210 including an indented feature that is complementary to a ball 1214 of the locking mechanism 1205. The locking mechanism 1205 includes a spring 1211 that pushes the ball 1214 into the indented feature of the implant connector 1210 to lock the implant 1200 to the catheter 1201. A pull wire 1213 (or suture) is coupled to a proximal end of the spring 1211. Pulling the pull wire 1213 causes the spring 1211 to release a force on the ball 1214, allowing the ball 1214 to move away from the intended feature of the implant connector 1210. Thus, pulling the pull wire 1213 causes the locking mechanism to transition from the locked configuration to the unlocked configuration. The catheter 1201 includes an inner lumen 1203 through which the pull wire 1213 runs from the locking mechanism 1205 to a proximal end of the catheter 1201. In other words, the implant connector 1210 (e.g., a connection mechanism) includes the ball 1214 in contact with the spring 1211 and the pull wire 1213 coupled to the spring 1211, the spring 1211 configured to bias a position of the ball 1214 to seat within an indentation formed in a proximal end of the implant 1200 (e.g., a sharpened coil), wherein pulling the pull wire 1213 adjusts the spring 1211 to allow the ball 1214 to move out of the indentation to release the implant 1200.

The disclosed puncturing devices and methods for creating fluid conduits between vessels can be used in conjunction with any suitable shunt. For example, after puncturing and dilating an opening between the left atrium and the coronary sinus using any of the devices and methods described herein, a shunt can be implanted in the dilated opening or fluid conduit that was created. Similarly, after puncturing and dilating an opening between the RPA and the SVC using any of the devices and methods described herein, a shunt can be implanted in the dilated opening or fluid conduit that was created.

The shunts, anchoring mechanisms, and/or puncturing mechanisms can be at least partially composed of any suitable material(s), which can include expandable stainless steel, cobalt chromium, textiles, and/or Nitinol. In some implementations, the disclosed anchoring or puncturing mechanisms, implants, and/or shunts may be configured to form a connection and/or bridge between two or more blood vessels and/or chambers. A shunt portion of an implant (such as a puncturing mechanism like a sharpened coil) may be configured to create and/or maintain a blood flow pathway between and/or through the tissue walls. In some examples, the disclosed shunts, puncturing mechanisms, and/or anchoring mechanisms may be at least partially composed of braided materials, which may include stainless steel, Nitinol, and/or other metals, polymers, and/or textile materials, including flexible and/or braided textiles. Textile materials can include memory-formed textiles. The disclosed implants may be configured to collapse to a smaller diameter for delivery while maintaining flexibility of the implant. In some examples, at least a portion of the implant may be covered by a tubular sheath (not shown) configured to surround at least a portion of the implant and/or the implant may comprise a solid tubular material. The sheath may be configured to prevent the implant from expanding from a crimped configuration. In some examples, additional and/or alternative devices and/or methods may be used to prevent expansion of the implant. For example, one or more wires may be attached to the implant to prevent expansion of the implant prior to delivery.

An example shunt for use between a left atrium and a coronary sinus can include an expandable stainless or cobalt chromium material. It can be expanded via coaxial displacement of a delivery catheter system. The shunt effective orifice area (EOA) and hence diameter are designed to achieve a reduction in pulmonary pressure while preserving the transpulmonary pressure gradient required to facilitate pulmonary perfusion and delivery of blood to the left atrium. The shunt EOA and length are designed to maintain this pressure reduction across a variety of clinical conditions, including but not limited to peripheral venous hypertension and exercise. The shunt includes a plurality of struts arranged longitudinal and circumferential with varying thicknesses and hence, mechanical properties. These variations in strut thickness and placement can be configured to facilitate expansion of the shunt body when, for example, an inner catheter body is moved with respect to an outer catheter body. For adjacent anatomic structures, the shunt can be established using two expandable elements. For non-adjacent structures, the shunt can be established using four expandable elements, two for each wall to effectively stabilize and prevent dislodgement of the shunt. The expandable segments can be deployed by withdrawing an inner rod towards the operator, causing deformation of the lower resistant expandable elements. The elements continue to expand until the non-shunt side of the expandable element comes into contact. Additional stabilization may be provided by flared barbs on the luminal facing surface of the expandable element that anchors the shunt to the tissue. In some implementations, anchoring with opposing expandable elements is included to fully capture both vascular walls. The shunt may be bare metal in the case of adjacent anatomic structures or covered in the case of non-adjacent anatomic structures. In some implementations, a plurality of expandable elements is included whereby the covered shunt segment inhibits or prevents infiltration of blood into the thoracic cavity. In some implementations, the shunt diameter can be expanded by continuing to approximate the expanders to radially deform the shunt segment.

In some implementations, the shunt is a self-expanding nitinol material that includes two primary components: a distal anchor and a proximal anchor. The distal anchor is attached to the shunt body. Once the distal anchor is unsheathed, a plurality of nitinol splines return to their lowest stress position of outward and retrograde deflection towards the proximal direction of the delivery system with an angle between 90-180 degrees. The distal anchor is then withdrawn slightly to capture the vascular wall via individual nitinol splines that terminate in a hook pattern. The proximal anchor is then deployed. The proximal anchor is not attached to the distal anchor containing shunt portion. This enables isolated vessel caval-pulmonary vessel approximation. Similar to the distal anchor, the proximal anchor is unsheathed and the plurality of nitinol splines deflect antegrade towards the distal end of the catheter. In some implementations, this proximal anchor is then advanced via the pusher to capture the second vessel wall. The proximal anchor is advanced to compress the two vessels together, creating a seal. In some implementations, advancement of the proximal anchor until it engages over the proximal shunt edge is provided whereupon it clips and locks into place. The distal and proximal anchors are able to bend to an adjustable thickness of RPA and/or SVC wall to maintain stability to the implant. In some implementations, the shunt diameter can be expanded once locked by compressing the distal and proximal anchors together in order to cause deformation of the shunt segment.

Certain shunts can be adaptive shunts. Such shunts include a gasket or pressure-dependent orifice regulator that modulates the amount of shunting to preferentially divert blood flow at higher pressures and minimize diversion at healthier pulmonary arterial pressures. In some implementations, the design reduces or minimizes cell attachment under non-dynamic or baseline conditions. This is accomplished by a pressure regulating system that is isolated from the vessel wall which minimizes migration of cells (e.g., myofibroblasts). Additionally, the geometry of the shunt is configured to promote fluid shear across both the vessel wall and shunt structure to minimize cell attachment from the circulating cells (e.g., fibroblasts). Accordingly, the adaptive shunts disclosed herein can be configured to deform under conditions of increased pressure gradient across the shunt. This is enabled by a “sail” like structure attached to a hinge comprised of a memory deformable material such as nitinol, with a distal segment section larger than the proximal section, representing varying conical, hexagonal, or pentagonal configurations. In some implementations, the sail is designed at the appropriate angle and percent of luminal cross-section area such that greater force is exerted under conditions of, for example, exercise or acute increases in pulmonary pressure that augment the pressure gradient to the SVC. Such varying level of deformation may be referred to as a pressure and flow “adaptive” shunt. In some implementations, an angle of the “sail” is used that promotes fluid shear across the surface, thereby reducing or minimizing the potential for circulating cell attachment. In some implementations, a circumferential portion of the “sail” that is not in contact with the cell wall is included, thereby reducing or minimizing the chance of cell migration and adhesion. This feature also enables a reduction in pressure at rest, which is further increased during elevations in the pulmonary-venous pressure gradient. In some implementations, an opening at the inflow portion of the sail apex (open circle, cross-section) is included that promotes relatively high fluid velocity and shear onto the endoluminal surface to prevent attachment and preserve device mobility.

Implantation of these various shunts can be performed percutaneously, under fluoroscopy and echocardiographic guidance. Given the different planes, simultaneous transthoracic or transesophageal echocardiographic guidance may be utilized. Various angulations may be utilized under fluoroscopy to guide implantation. In some implementations, the internal jugular vein and the right femoral vein can be used for dual access. In some implementations, the shunt is placed at the end of a transcatheter delivery system which traverses the right atrium, right ventricle, into the right pulmonary artery. This delivery catheter may or may not have an end hole or side hole catheter to inject contrast to confirm location. The catheter, in some implementations, can have one or more articulation points at the distal end of the catheter to allow for manipulation and angulation, with a needle at the distal end for puncture. In such implementations, there may be a loop or a snare marker in the SVC to allow for targeted puncture, and or capture of the distal end or wire as needed. The device can be extended across the RPA-SVC to be placed and create a shunt between the RPA to SVC.

In some implementations, coiled wire, snare, or wire marker may be used to traverse the right atrium, right ventricle, and right pulmonary artery, with imaging guidance. The imaging guidance may come in the form of a catheter with end or side hole contrast angiography, with a radio-opaque tip, or an echogenic tip, or a combination of the above. This marker would mark the RPA site. The delivery catheter can then be utilized in the SVC from either the femoral vein or the internal jugular vein with one or more articulation points at the distal end of the delivery catheter to facilitate targeted puncture of the SVC-RPA. With adjunct imaging guidance, the SVC and RPA can be punctured with subsequent placement of the device and creation of the shunt.

### Additional Features and Embodiments

The present disclosure provides methods and devices (including various medical implants) for shunting blood within a human body. The term “implant” is used herein according to its plain and/ordinary meaning and may refer to any medical implant, frame, valve, shunt, stent, anchor, and/or similar devices for use in treating various conditions in a human body. Implants may be delivered via catheter (i.e., transcatheter) for various medical procedures and may have a generally sturdy and/or flexible structure. The term “catheter” is used herein according to its broad and/ordinary meaning and may include any tube, sheath, steerable sheath, steerable catheters, and/or any other type of elongate tubular delivery device comprising an inner lumen configured to slidably receive instrumentation, such as for positioning within an atrium or coronary sinus, including for example delivery catheters and/or cannulas. Some transcatheter processes described herein can utilize a single catheter or multiple catheters.

Although certain preferred embodiments and examples are disclosed below, inventive subject matter extends beyond the specifically disclosed embodiments to other alternative embodiments and/or uses, and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The above description of embodiments of the disclosure is not intended to be exhaustive or to limit the disclosure to the precise form disclosed above. While specific embodiments, and examples, are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel or may be performed at different times.

Certain terms of location are used herein with respect to the various disclosed embodiments. Although certain spatially relative terms, such as “outer,” “inner,” “upper,” “lower,” “below,” “above,” “vertical,” “horizontal,” “top,” “bottom,” and similar terms are used herein to describe a spatial relationship of one device/element or anatomical structure relative to another device/element or anatomical structure, it is understood that these terms are used herein for ease of description to describe the positional relationship between element(s)/structures(s), as illustrated in the drawings. Spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited. In some contexts, description of an operation or event as occurring or being performed “based on,” or “based at least in part on,” a stated event or condition can be interpreted as being triggered by or performed in response to the stated event or condition.

With respect to the various methods and processes disclosed herein, although certain orders of operations or steps are illustrated and/or described, it should be understood that the various steps and operations shown and described may be performed in any suitable or desirable temporal order. Furthermore, any of the illustrated and/or described operations or steps may be omitted from any given method or process, and the illustrated/described methods and processes may include additional operations or steps not explicitly illustrated or described.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the subject matter herein disclosed and claimed below should not be limited by the particular embodiments described above but should be determined only by a fair reading of the claims that follow.

Unless the context clearly requires otherwise, throughout the description and the claims, the terms “comprise,” “comprising,” “have,” “having,” “include,” “including,” and the like are to be construed in an open and inclusive sense, as opposed to a closed, exclusive, or exhaustive sense; that is to say, in the sense of “including, but not limited to.”

The word “coupled”, as generally used herein, refers to two or more elements that may be physically, mechanically, and/or electrically connected or otherwise associated, whether directly or indirectly (e.g., via one or more intermediate elements, components, and/or devices. Additionally, the words “herein,” “above,” “below,” and words of similar import, when used in this application, shall refer to this application as a whole, including any disclosure incorporated by reference, and not to any particular portions of the present disclosure. Where the context permits, words in present disclosure using the singular or plural number may also include the plural or singular number, respectively.

The word “or” in reference to a list of two or more items, that word covers all of the following interpretations of the word: any of the items in the list, all of the items in the list, and any combination of the items in the list. Furthermore, as used herein, the term “and/or” used between elements (e.g., between the last two of a list of elements) means any one or more of the referenced/related elements. For example, the phrase “A, B, and/or C” means “A,” “B,” “C,” “A and B,” “A and C,” “B and C,” or “A, B, and C.”

As may be used herein, the terms “substantially” and “approximately” provide an industry-accepted tolerance for its corresponding term and/or relativity between items. For some industries, an industry-accepted tolerance is less than one percent, while for other industries, the industry-accepted tolerance may be 10 percent or more. Other examples of industry-accepted tolerances range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than approximately +/−1%). Some relativity between items may range from a difference of less than a percentage level to a few percent. Other relativity between items may range from a difference of a few percent to magnitude of differences.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same, related, or unrelated reference numbers. The relevant features, elements, functions, operations, modules, etc. may be the same or similar functions or may be unrelated.

What is claimed is:

1. A puncture device to facilitate puncturing through a wall of a second vessel from a first vessel, the puncture device comprising:
a catheter configured to be manipulated;
a sharpened coil coupled to a distal end of the catheter; and
a needle adapted to extend from the distal end of the catheter approximately through a central axis of the sharpened coil,
wherein manipulating the catheter causes the sharpened coil to rotate to drive the sharpened coil through a wall of the first vessel and into the wall of the second vessel,
wherein the sharpened coil anchors the catheter to the first vessel and the second vessel to facilitate puncturing from the first vessel to the second vessel with the needle.

2. The puncture device of claim 1 further comprising a dilation device that is adapted to open the wall of the first vessel and the wall of the second vessel to create a conduit between the first vessel and the second vessel.

3. The puncture device of claim 1, wherein the sharpened coil is detachable from the catheter.

4. The puncture device of claim 3 further comprising a release wire threaded through a proximal opening formed in the sharpened coil.

5. The puncture device of claim 4, wherein the release wire secures the sharpened coil to the catheter by being threaded through the proximal opening.

6. The puncture device of claim 4, wherein the sharpened coil is adapted to be released from the catheter by pulling the release wire proximally to unthread the release wire from the proximal opening formed in the sharpened coil.

7. The puncture device of claim 3 wherein the catheter includes a connection mechanism comprising a ball in contact with a spring and a pull wire coupled to the spring, the spring adapted to bias a position of the ball to seat within an indentation formed in a proximal end of the sharpened coil, wherein pulling the pull wire adjusts the spring to allow the ball to move out of the indentation to release the sharpened coil.

8. The puncture device of claim 1, wherein the sharpened coil includes a pull wire attached to a distal end of the sharpened coil such that a proximal force on the pull wire causes the sharpened coil to compress.

9. The puncture device of claim 1, wherein a distal end and a proximal end of the sharpened coil each have a larger radius than a middle portion of the sharpened coil between the distal end and the proximal end.

10. A puncture device to facilitate puncturing through a wall of a second vessel from a first vessel, the puncture device comprising:
a sharpened coil configured to be driven through a wall of the first vessel and the wall of the second vessel;
a needle adapted to extend from a distal end of a catheter through a central axis of the sharpened coil, wherein the sharpened coil anchors the catheter to the first vessel and to the second vessel to facilitate puncturing through a wall of the first vessel and the wall of the second vessel with the needle; and
a dilation device that is adapted to open the wall of the first vessel and the wall of the second vessel and to create a conduit between the first vessel and the second vessel.

11. The puncture device of claim 10 further comprising a release wire threaded through a proximal opening formed in the sharpened coil to secure the sharpened coil to a delivery device.

12. The puncture device of claim 11, wherein the sharpened coil is adapted to be released from the delivery device by pulling the release wire proximally to unthread the release wire from the proximal opening formed in the sharpened coil.

13. The puncture device of claim 10 further comprising a connection mechanism to secure the sharpened coil to a delivery device, the connection mechanism comprising a cylindrical housing forming an opening adapted to secure a proximal end of the sharpened coil, the proximal end of the sharpened coil forming a spatula-shaped proximal leg that fits through a slot in the cylindrical housing so that, when secured to the delivery device by the connection mechanism, a wide portion of the spatula-shaped proximal leg sits within the opening and a narrow portion of the spatula-shaped proximal leg sits within the slot.

14. The puncture device of claim 12, wherein the sharpened coil is adapted so that the spatula-shaped proximal leg is configured to rotate to be released from the connection mechanism.

15. The puncture device of claim 12, wherein the connection mechanism is configured to open to increase a size of the slot to release the sharpened coil from the connection mechanism.

16. The puncture device of claim 10 further comprising a connection mechanism comprising a ball in contact with a spring and a pull wire coupled to the spring, the spring adapted to bias a position of the ball to seat within an indentation formed in a proximal end of the sharpened coil, wherein pulling the pull wire adjusts the spring to allow the ball to move out of the indentation to release the sharpened coil.

17. A puncture device to facilitate puncturing through a wall of a second vessel from a first vessel, the puncture device comprising:
a sharpened coil configured to be driven through a wall of the first vessel and the wall of the second vessel; and
a needle adapted to extend from a distal end of a catheter through a central axis of the sharpened coil, wherein the sharpened coil anchors the catheter to the first vessel and to the second vessel to facilitate puncturing through a wall of the first vessel and the wall of the second vessel with the needle,
wherein the sharpened coil is detachable from the catheter.

18. The puncture device of claim 17 further comprising a release wire threaded through a proximal opening formed in the sharpened coil to secure the sharpened coil to a delivery device.

19. The puncture device of claim 18, wherein the sharpened coil is adapted to be released from the delivery device by pulling the release wire proximally to unthread the release wire from the proximal opening formed in the sharpened coil.

20. The puncture device of claim 17 further comprising a connection mechanism comprising a ball in contact with a spring and a pull wire coupled to the spring, the spring adapted to bias a position of the ball to seat within an indentation formed in a proximal end of the sharpened coil, wherein pulling the pull wire adjusts the spring to allow the ball to move out of the indentation to release the sharpened coil.
